(19) United States
(12) Patent Application Publication
(10) Pub. No.: US 2010/0023056 A1
(43) Pub. Date: Jan. 28, 2010

(54) TETHER-ANCHOR ASSEMBLIES

(75) Inventors: Peter K. JOHANSSON, Lafayette, CA (US); Marshall TSURUDA, San Jose, CA (US)

Correspondence Address: MORRISON & FOERSTER LLP, 755 PAGE MILL RD, PALO ALTO, CA, 94304-1018, US

(73) Assignee: Guided Delivery Systems Inc., Santa Clara, CA (US)

(21) Appl. No.: 12/505,332

(22) Filed: Jul. 17, 2009

Related U.S. Application Data
(60) Provisional application No. 61/083,109, filed on Jul. 23, 2008.
(60) Provisional application No. 61/160,018, filed on Mar. 13, 2009.

(51) Int. Cl. A61B 17/04

(52) U.S. Cl. 606/232; 29/428

(57) ABSTRACT

Anchors, tethers, knot configurations and other coupling mechanisms used to secure tethers to anchors or other implants are described. The knot configurations described may provide enhanced knot security and/or tensile strength, while also occupying relatively little volume. In some variations, the surfaces of a tether and/or an anchor or other implant may be at least partially coated with one or more agents and/or may be at least partially textured.

### CROSS-REFERENCE TO RELATED APPLICATIONS

The present application claims priority under 35 U.S.C. §119(e) to U.S. Provisional Application No. 61/083,109 and was filed on Jul. 23, 2008 and U.S. Provisional Application No. 61/160,018 and was filed on Mar. 13, 2009, which are hereby incorporated by reference in their entirety.

### TECHNICAL FIELD

The devices and methods described herein relate generally to knot and other configurations for coupling tether-anchor assemblies. The configurations described here may be particularly useful for securing tethers, such as sutures, to anchors or other implants.

### BACKGROUND

Tether-anchor assemblies are often used to join tissues or to attach material (e.g., a prosthetic valve) to tissue. Tissues may be joined, for example, to close wounds and/or to modify body structures. In some cases, tissues may be joined during a transplant or tissue-grafting procedure within the body, such as within the heart. As an example, a procedure for reconfiguring annular tissue (e.g., mitral valve annular tissue) may comprise implanting a plurality of anchors that are coupled to a common tether into the tissue.

### BRIEF DESCRIPTION OF THE DRAWINGS

FIG. 1A is a side perspective view of a variation of an anchor.

FIG. 1B is an illustration of a variation of a tether.

FIG. 2A is an illustration of another variation of a tether.

FIG. 2B is an illustration of an additional variation of a tether.

FIGS. 3A-3H depict a variation of a method for tying a tether to an anchor using a bowline knot.

FIG. 4A shows a variation of a bowline knot used to secure a tether to an anchor.

FIG. 4B shows another variation of a bowline knot used to secure a tether to an anchor.

FIGS. 4C and 4D show a tether that has been secured to an anchor using a variation of a bowline knot, during different stages of tightening of the tether.

FIGS. 5A-5F depict a variation of a method for securing an anchor to a tether using a figure-of-eight knot.

FIGS. 6A-6D illustrate a variation of a method for securing an anchor to a braided tether using an eye splice.

FIGS. 7A-7H depict a variation of a method for tying a back splice in a braided tether.

FIG. 8 shows a braided tether secured to an anchor by a back splice.

FIG. 9 is a cross-sectional view of a heart with a variation of an anchor delivery device being positioned for treatment of annular tissue (as shown, mitral valve annular tissue).

FIGS. 10A-10F illustrate a variation of a method for applying anchors to annular tissue and cinching the anchors to tighten the annular tissue, using a variation of an anchor delivery device.

FIG. 11 depicts another variation of a tether-anchor assembly.

FIG. 12 depicts another variation of a tether-anchor assembly.

FIG. 13 depicts another variation of a tether-anchor assembly.

FIG. 14 depicts another variation of a tether-anchor assembly.

FIG. 15 depicts another variation of a tether-anchor assembly.

FIG. 16 depicts another variation of a tether-anchor assembly.

FIG. 17 depicts another variation of a tether-anchor assembly.

FIG. 18 depicts another variation of a tether-anchor assembly.

FIGS. 19A to 19D schematically illustrate a method of forming a knot using a tether attached to an anchor.

### BRIEF SUMMARY

Described here are tether-anchor assemblies and related methods. In some variations, the tether-anchor assemblies include at least one knot configuration that couples a tether to one or more anchors and/or other implants. Examples of suitable knot configurations include bowline knots, figure-of-eight knots, splices (e.g., eye splices, back splices, etc.), and the like. Some variations of the knot configurations may exhibit relatively high knot security, reduced tether material stress, and/or tensile strength, while also occupying a relatively small knot volume.

Certain variations of the tether-anchor assemblies comprise two anchors, each of which comprises an eyelet region and a penetrating region configured to penetrate a tissue. The tether-anchor assemblies further comprise a tether slidably coupled to one of the eyelet regions and fixedly coupled to the other eyelet region via a knot assembly. The knot assembly comprises one or more knots, such as, for example, one or more bowline knots, figure-of-eight knots, eye splices, and/or back splices.

The knot assembly may comprise just one knot, or may comprise multiple (e.g., two, three, four, five, etc.) knots. For example, the knot assembly may comprise both a bowline knot and a figure-of-eight knot. In some variations, the bowline knot may be located between the eyelet region of the fixedly coupled anchor and the figure-of-eight knot. In other variations, the figure-of-eight knot may be located between the eyelet region of the fixedly coupled anchor and the bowline knot. When the knot assembly comprises multiple knots, at least some of the knots may be the same as each other, or all of the knots may be different from each other.

At least one of the anchors may be configured to self-expand. In some variations, at least a portion of at least one of the anchors may be coated. Examples of coating materials include Vascular Endothelial Growth Factor, Fibroblast Growth Factor, Platelet-Derived Growth Factor, Transforming Growth Factor Beta, insulin, insulin-like growth factors, estrogens, heparin, and Granulocyte Colony-Stimulating Factor. Other suitable coating materials may alternatively or additionally be used. In certain variations, at least a portion of at least one of the anchor eyelet regions may comprise a surface that is textured to enhance the surface's coefficient of friction. While anchors have been described, knots may also be used to couple tethers to other types of implants, such as leads or electrodes.

The tether may be a monofilament tether or a multifilament tether. In some variations, the tether may be braided. In certain variations, at least a portion of the tether may comprise a surface that is textured to enhance the surface's coefficient of friction. The tether may be made from one or more biodegradable materials and/or non-biodegradable materials. Moreover, at least a portion of the tether may be coated (e.g., with one or more agents). Non-limiting examples of suitable coating materials include wax, silicone, silicone rubbers, PTFE, PBA, and ethyl cellulose.

In other variations of the tether-anchor assemblies, an anchor may be inserted or otherwise located in an opening within the elongate body of the tether. For example, an anchor may be passed through the strands or fibers of a multi-filament tether, or other openings provided or formed in a tether. In another example, an eyelet structure may be crimped to a tether. In still other examples, the anchor may be crimped to the tether. In some variations, the anchor may be configured to limit or restrict sliding or other movement with respect to the tether. The tether may also be heat-set or otherwise treated to at least partially adhere the fibers of the tether together, or to stiffen a segment of the tether, for example. In some variations, treatment of the tether may resist separation or loosening of the strands or fibers when the tether is tensioned.

In one variation, a tether-anchor assembly is provided, comprising a tether comprising multiple filaments and having a distal end and an opening formed between the multiple filaments, wherein at least a portion of the multiple filaments between the distal end and the opening are heat-fused, and an anchor with an eyelet segment slidably located within the opening of the tether and a penetrating segment configured to penetrate a tissue.

In another variation, a tether-anchor assembly is provided, comprising a tether comprising a proximal end, a distal end, an elongate body therebetween, and an intrabody opening, and an anchor with an eyelet segment located within the intrabody opening of the tether and a penetrating segment configured to penetrate a tissue. In some variations, the tether is a multi-filament tether, and may comprise at least six or eight strands in some examples. In some further examples, at least three strands are located on opposite sides of the intrabody opening. In other variations, the tether may be a monofilament tether. The tether may be heat-treated at least between the distal end and the intrabody opening. The tether may further comprise a stop region between the distal end and the intrabody opening, including a fused stop region. In variations comprising multiple filaments, the multiple filaments of the tether may be at least partially fused between the distal end and the intrabody opening. In some examples, the tether may comprise polyethylene, such as ultra-high molecular weight polyethylene.

In another variation, a method for making a tether-anchor assembly is provided, comprising inserting a multi-filament tether into a protective tube, heat-treating the multi-filament tether using the protective tube, separating at least some filaments of the tether, and passing an anchor through the separated filaments. In some examples, separating at least some filaments of the tether may comprise equally separating the filaments between a first bundle and a second bundle, or separating the filaments such that the number of filaments in the first bundle and the second bundle are no greater than one filament.

### DETAILED DESCRIPTION

Certain knot configurations provide inadequate security and/or exhibit weak tensile strength. Moreover, some knot configurations have a relatively large volume, and thus may occupy a relatively large amount of space at a securing site. Accordingly, it would be desirable to provide an improved knot configuration for securing a tether to an anchor, in which the knot configuration is relatively secure and/or has a relatively high tensile strength. It would also be desirable for the knot configuration to occupy minimal volume.

Described here are various configurations used to secure tethers to anchors to form tether-anchor assemblies. While anchors are described, the devices, methods, and configurations described herein may be applied to any other suitable implants, such as leads or electrodes, or any other implants capable of fixedly securing a tether to body tissue. Additionally, the devices, methods, and knot configurations may be used in any suitable medical procedure (e.g., in the fields of general surgery, cardiology, neurosurgery, gastroenterology, and the like).

Certain variations of the tether-anchor assemblies described herein comprise a plurality of anchors coupled to a common tether. Some of the anchors are slidably coupled to the tether, while others are fixedly coupled to the tether using, for example, one or more knot configurations, such as bowline knots, figure-of-eight knots, eye splices, and/or back splices. Other appropriate knot configurations may also be used. In some variations, the anchors may be attached to annular tissue of the heart. For example, the anchors may be attached to mitral valve annular tissue, and the tether may be tensioned to cause cinching of the mitral valve annulus. It should be noted that while one tether has been described, the devices, methods, and knot configurations described here may be applied to multiple (e.g., two, three, four, five, etc.) tethers that are, for example, used together in a single procedure. The multiple tethers may be attached to a single anchor, or some or all of the tethers may each be attached to different anchors.

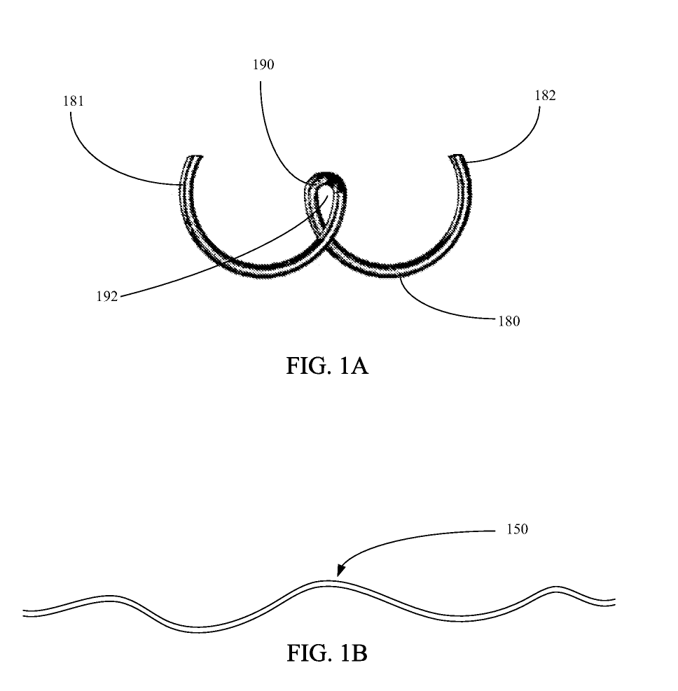

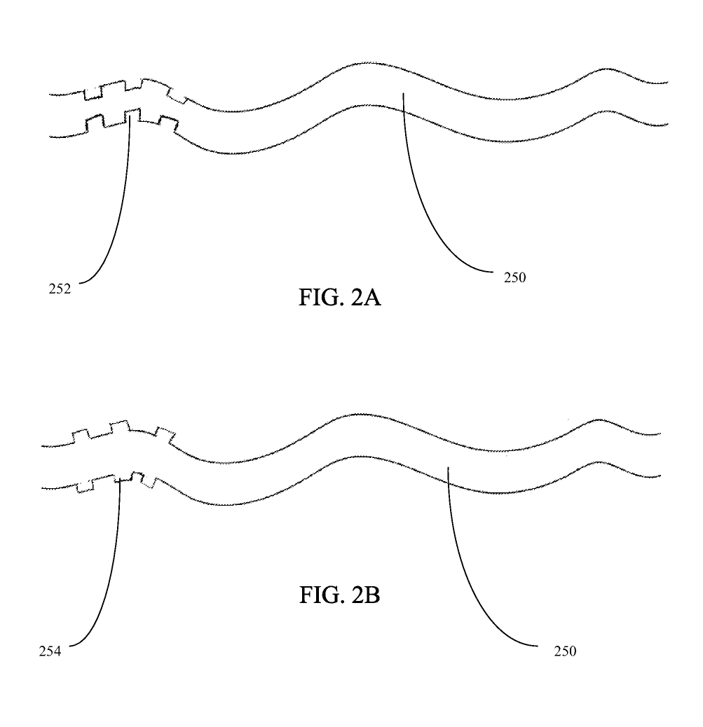

Turning now to the figures, FIG. 1A shows a variation of an anchor (180). As shown, anchor (180) comprises two curved legs (181) and (182) that are adapted to penetrate tissue. Legs (181) and (182) cross to form a loop corresponding to an eyelet region (190) defining an eyelet (192).

In the variation shown in FIG. 1A, legs (181) and (182) cross once to form eyelet region (190), which is continuous with legs (181) and (182), and is substantially centered between legs (181) and (182). However, some variations of anchors may comprise legs and one or more eyelet regions that are not centered between the legs. Moreover, anchors may comprise legs having different lengths and/or shapes. Additionally, certain variations of anchors may comprise an eyelet region having more than one loop. As an example, an eyelet region may have a helical shape including more than one loop (e.g., two loops, three loops, etc.). In some variations, an eyelet region may comprise a partial loop, such as half-loop. Different types of anchors and other implants are described in further detail below.

FIG. 1B shows a variation of a tether (150). Any suitable tether may be used, and the type of tether that is used may depend, in some cases, on the application. In certain variations, the tether may comprise a suture or suture-like material. Moreover, while tether (150) is shown as a single piece of material, the tether may also be formed of two or more pieces of material. Tethers may comprise any suitable material or materials. Non-limiting examples of suitable materials include nylon, polymers such as polyesters (e.g., DACRON® polyester strips), polyethylenes (e.g., ultra-high molecular weight polyethylene), polyglycolic acid (PGA), polylactic acid (PLA), and other suitable polymers, and metals or metal alloys such as nickel-titanium alloys (e.g., Nitinol®), stainless steel, cobalt-chromium alloys, and other suitable metal alloys. In some variations of tethers, combinations of different types of materials may be used, such as a combination of a polymer (e.g., a polyester) and a metal alloy (e.g., Nitinol®), or a combination of at least two different polymers and/or at least two different metal alloys. Any other suitable materials may also be used. In certain variations, tethers may be formed of multiple yarns and/or fibers of the aforementioned material or materials, braided in such a manner as to provide target performance characteristics, such as tensile strength, stiffness, abrasion resistance, and/or mechanical patency. Different types of tethers are described in further detail below.

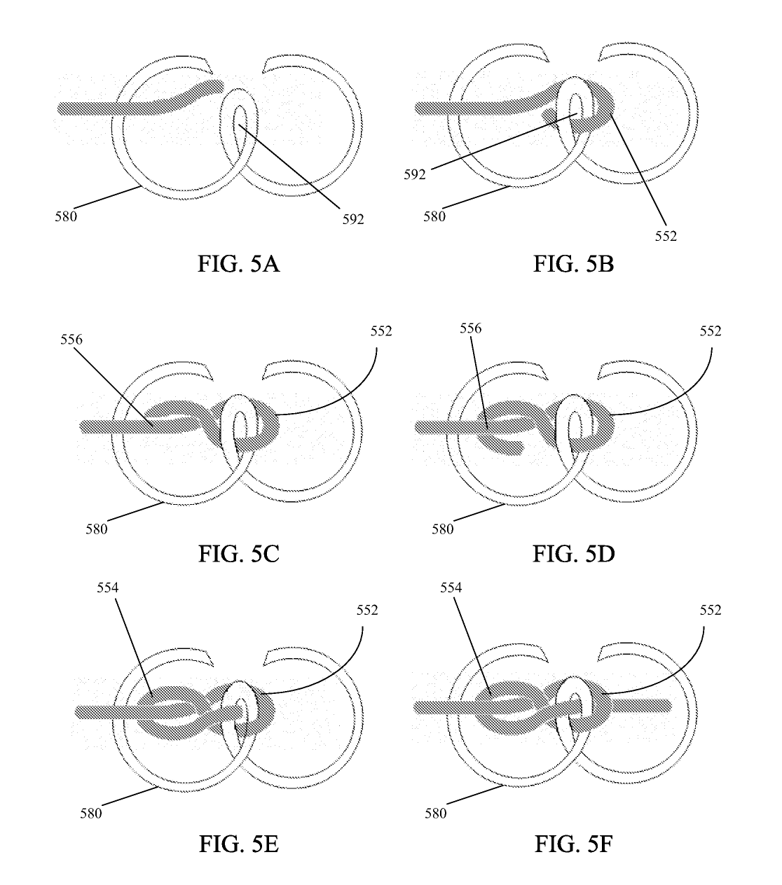

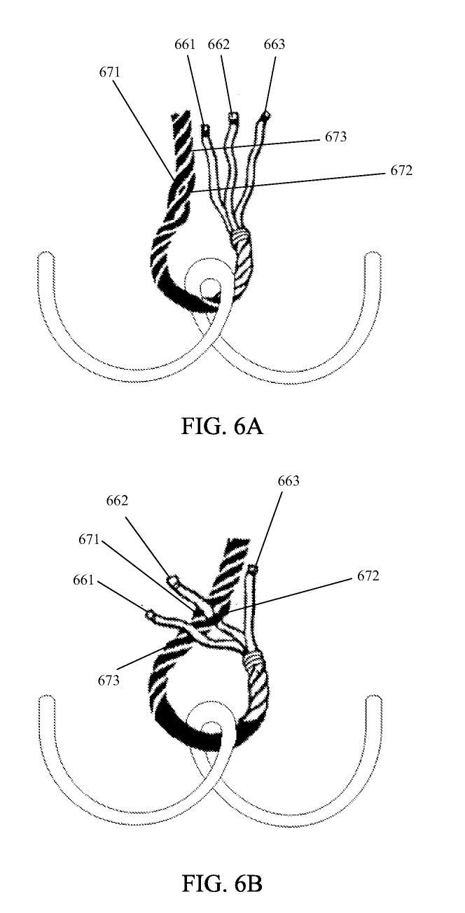

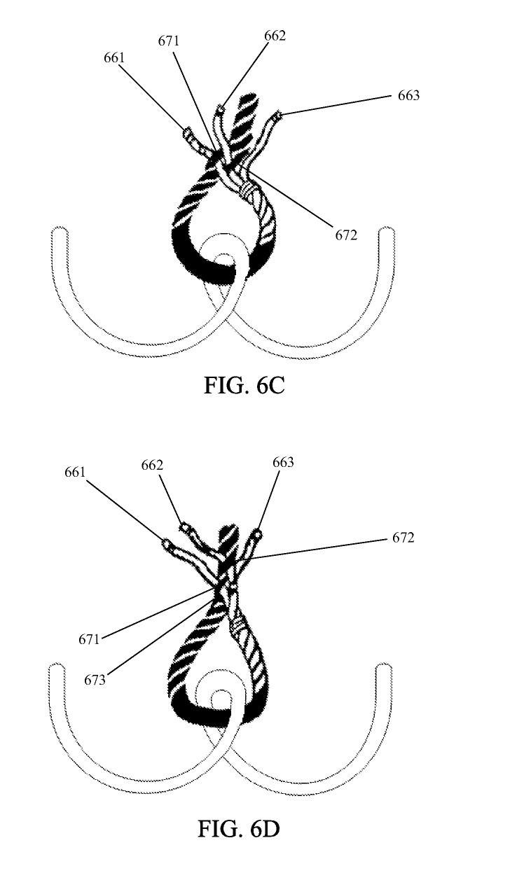

As described above, one or more tethers may be coupled to one or more anchors, thereby forming a tether-anchor assembly. The tethers may be coupled to the anchors using, for example, one or more knot configurations, and/or any other appropriate coupling mechanism. For example, FIG. 4A shows a variation of a tether-anchor assembly (400) comprising an anchor (480) coupled to a tether (450) via a bowline knot (410). FIG. 4C shows bowline knot (410) in a loose (untightened) state, and FIG. 4D shows bowline knot (410) in a tightened state.

A tether knot, such as bowline knot (410), is a fastening formed by intertwining various segments of the tether. Some variations of knots may include one or more loop portions, knot portions, and/or end portions (often also referred to as tails or ears). Referring to FIG. 4A for example, bowline knot (410) comprises a loop portion (412), a knot portion (414), and an end portion (416). Loop portion (412) is formed of the segment of tether (450) that passes through the eyelet of anchor (480) and engages the eyelet region of anchor (480). Knot portion (414) comprises the portion of tether (450) in which multiple tether segments are intertwined. End portion (416), as shown, includes one of the end segments of tether (450). The length of end portion (416) is relatively short (which helps reduce knot volume), but is sufficient to limit the likelihood of bowline knot (410) becoming loose or untied through slippage. In certain variations, end portion (416) may have a length that is from about 0.1 mm to about 5 mm, sometimes from about 0.2 mm to about 3 mm, and sometimes from about 1 mm to about 2 mm. While end portion (416) is relatively short, certain variations of knot configurations may include one or more end portions that are relatively long.

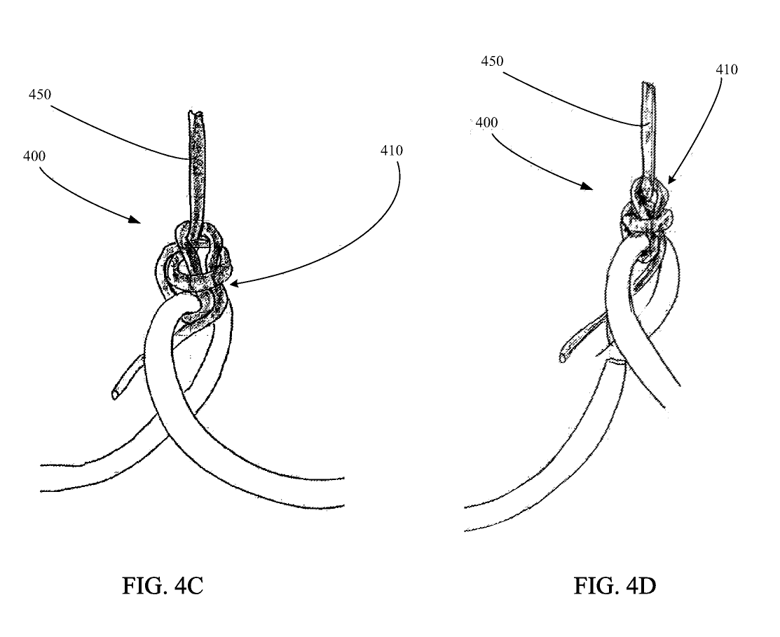

FIGS. 3A-3H depict a variation of a method for forming a bowline knot to couple a tether to an anchor. As shown in FIGS. 3A-3C, a first loop (352) is formed from a portion of a tether (350). Next, and as shown in FIGS. 3C and 3D, the end of the tether is advanced through an eyelet (392) of an anchor (380). Referring now to FIGS. 3D-3H, the tether end may then be advanced through first loop (352), thereby forming a second loop (354), passed around a tether portion (356), and then advanced back through first loop (352). Thereafter, the knot may be tightened. The resulting tether-anchor assembly may be similar or identical to the tether-anchor assembly illustrated in FIG. 4A.

While a variation of a bowline knot is shown in FIGS. 4A-4D, other variations of bowline knots, or other types of knot configurations, may also be used. Factors that may be considered in determining which knot configuration is appropriate for forming a tether-anchor assembly may include, for example, knot security, tensile strength, tether knot stress, and/or knot volume. For some applications, an ideal knot configuration may be one that exhibits high knot security and tensile strength, while also having minimal knot volume.

Knot security corresponds to the effectiveness of the knot in resisting slippage. If knot security in a tether-anchor assembly is inadequate, knot loosening may occur. As a result, the tether may decouple from the anchor. Depending on the application, it may be desirable to have relatively high knot security. As an example, knots used in conjunction with an anchor to secure constantly moving tissue, such as heart valve tissue, may require greater knot security than those that are used in conjunction with an anchor to secure non-moving tissue.

Generally, as frictional forces between the tether segments in a knot increase, knot security can also increase. The amount of frictional forces between the tether segments in a knot depends in part on the coefficient of friction of the tether material, the particular knot configuration used, and the initial tension applied during knot tying. Multifilament tethers typically have greater coefficients of friction than their monofilament counterparts. Accordingly, knot configurations formed of one or more multifilament tethers may exhibit better knot security and less slippage than knot configurations formed of one or more monofilament tethers.

In addition to the above-described factors, knot security may also be enhanced by using a tether having a relatively large diameter, and/or by using a knot configuration comprising additional loops (also often referred to as ties or throws) passing through the anchor eyelet or eyelets, or through or around other parts of the anchor. Typically, the knot security for a given type of knot configuration can increase with an increasing number of throws in the knot configuration. In some cases, however, a maximum knot security level may be reached, beyond which further throws have minimal or no effect on knot security.

The tensile strength of a tether is the amount of force necessary to break the tether. The relative tensile strengths of different tethers may be measured, for example, using the ASTM D-2256-02 Test Method for Tensile Properties of Yarns by the Single-Strand Method. Other testing methods may be used, including, for example, testing methods that are independently developed for unique applications. The tensile strength of a tether depends in part on the material used in the construction of the tether, the size of the tether, the method used to form the tether, and/or environmental factors, such as the age of the tether. When the tether includes at least one knot, the tensile strength of the tether depends in part on the number of throws in the knot. The tensile strength of a knotted tether generally corresponds to the knot strength.

Knot volume is equal to the space occupied by the knot. A knot having a relatively large volume generally has a relatively large profile, which may present several disadvantages. First, a large-volume knot may have an increased risk of contact with tissue, relative to a small-volume knot. Contact between the knot and the tissue may increase the risk of tissue damage, delay the healing process, and/or increase the risk of infection. Second, in situations where the knot is located within a bloodstream pathway (e.g., in a blood vessel or inside a heart), a large-volume knot may disrupt laminar blood flow and create turbulence, which can negatively impact a patient's blood circulation. Third, a large-volume knot may present fitting issues with regard to the anchor's eyelet. In view of the above considerations, it may be desirable to use a knot having a relatively small volume in certain instances. However, knots having a large knot volume may also be desirable in certain instances and are contemplated here. For example, in some instances knots having a large knot volume are desirable for increased knot security.

Additional non-limiting examples of knot configurations are described below. The following knot configurations may exhibit relatively high knot security and/or tensile strength. They may also occupy a relatively small volume.

While one variation of a bowline knot has been described above with reference to FIGS. 4A-4D, other bowline knot variations may also be used for forming tether-anchor assemblies as described here. As previously described, in the bowline knot-forming method shown in FIGS. 3A-3H, a tether end is advanced through first loop (352), then directed toward the left (with respect to the perspective provided by the schematic drawing) across the back of tether portion (356), and then through first loop (352) again. FIG. 4A shows an example of a tether-anchor assembly that may be formed using this method. However, other variations of suitable methods may also be used. For example, in an alternative variation of a method for forming a bowline knot, the tether end may be directed toward the right (rather than the left) across the back of tether portion (356), and then back through first loop (352). An example of a tether-anchor assembly that may be formed using this method is shown in FIG. 4B.

FIGS. 4A and 4B are illustrative of another variation between different knot configurations. As shown in FIG. 4A, in some variations the end portion of a tether may be advanced through an anchor eyelet. This may, for example, provide a reduced overall knot profile, and/or may improve the security of the knot (e.g., by limiting the likelihood of undesired twisting of the end portion). However, and as shown in FIG. 4B, in other variations, the end portion of a tether may not be advanced through an anchor eyelet. Of course, it should be understood that in variations of tether-anchor assemblies where the anchor has no eyelet, the knot may be formed at any suitable location along the anchor.

Bowline knots may exhibit a number of different advantages. For example, some variations of bowline knots may provide enhanced knot security and/or tensile strength relative to conventional knots used to couple a tether to an anchor. The enhanced knot security may allow for only one bowline knot to be used to secure a tether to an anchor in a medical procedure. This may be advantageous, for example, because a reduction in the number of knots used to secure a tether to an anchor may also result in a reduction in total knot volume. Additionally, in certain variations, a bowline knot may have a knot volume that is smaller than the volume of a corresponding conventional knot (as measured by the volume required to reach a certain level of knot security).

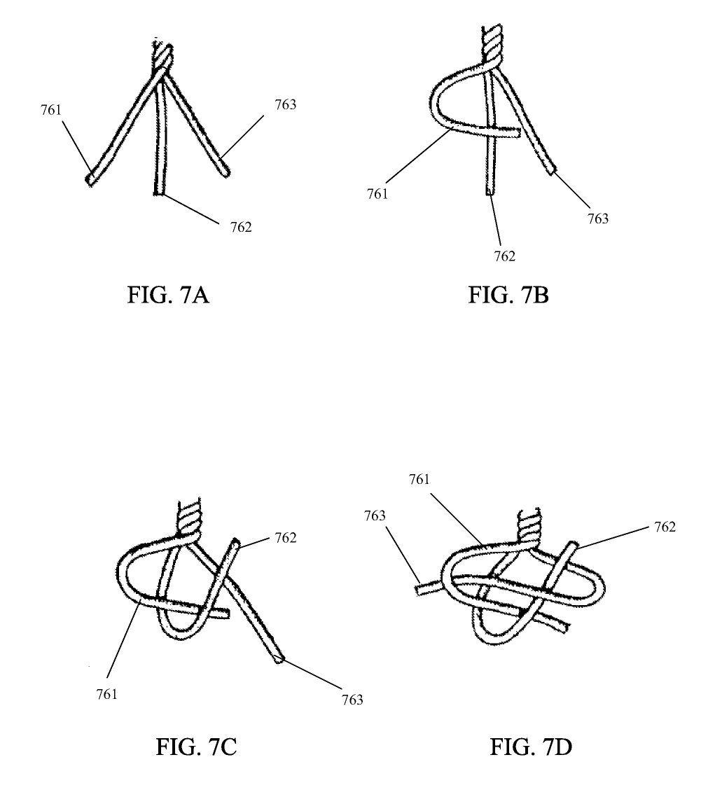

In some variations, a knot having a figure-of-eight configuration may be used to couple a tether to an anchor. FIGS. 5A-5F depict a variation of a method for coupling a tether to an anchor using a figure-of-eight knot. As shown in FIGS. 5A and 5B, a tether end is advanced through an eyelet (592) of an anchor (580), and a first loop (552) is formed from a segment of the tether. Thereafter, and as shown in FIGS. 5C and 5D, the tether end is advanced under a tether portion (556). Referring now to FIGS. 5D-5F, the tether end is then advanced through first loop (552), thereby creating a second loop (554). The resulting knot may then be tightened. Although FIGS. 5B-5F show anchor (580) coupled to first loop (552) of the figure-of-eight knot, in certain variations, anchor (580) may be coupled to second loop (554).

In some variations, a knot assembly comprising two or more different types of knot configurations may be used to couple a tether to an anchor. As an example, a tether may be secured to an anchor by first tying a bowline knot in the tether, and then tying a figure-of-eight knot in the tether. As another example, in certain variations, the knot tying order may be reversed, such that a figure-of-eight knot is tied first, followed by a bowline knot. In some variations, a tether may be secured to an anchor by more than two knots (e.g., three, four, five, or six knots, etc.), and the knots may include bowline knots, figure-of-eight knots, any other suitable knots, or combinations thereof. Moreover, certain variations of tether-anchor assemblies may comprise one or more of the knots described herein, in combination with one or more conventional knots.

Another example of a type of knot that may be used to couple a tether to an anchor is a splice, which may be formed by interweaving tether filament strands. When a tether has a splice in it, the tether comprises a standing portion and a splice portion. In some variations, a splice may provide greater overall tether tensile strength than another type of knot. Furthermore, splices may be relatively compact and may therefore occupy a relatively small volume of space. Exemplary splices that may exhibit these features are described in further detail below.

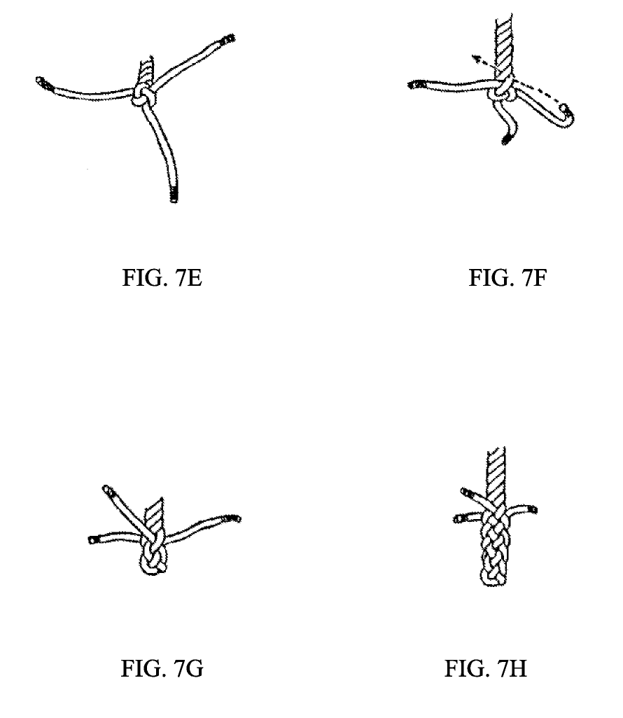

In some variations, a tether may be coupled to an anchor via an eye splice. An eye splice may be formed by weaving the ends of fi lament strands of a tether back into the tether, to form a loop or eye. In certain variations, a tether may be tied to an anchor using an eye splice as follows. First, the length of the tether segment that will form the eye portion is determined. Next, and as shown in FIG. 6A, the tether end is passed through an anchor eyelet (or through or around another anchor portion) and unwound so that the filament strands are spread apart. Thereafter, each filament strand is woven back into a portion of the tether that is proximal to the ends of the filament strands. For example, FIGS. 6A-6D show strands (661), (662), and (663) being woven back into strands (671), (672), and (673). Strands (661), (662), and (663) may then be pulled and tightened, and the weaving process may be repeated until a sufficient tether length has been tucked.

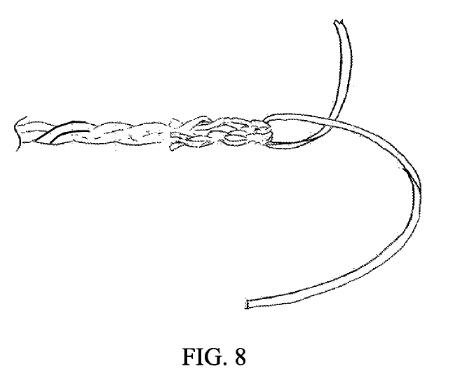

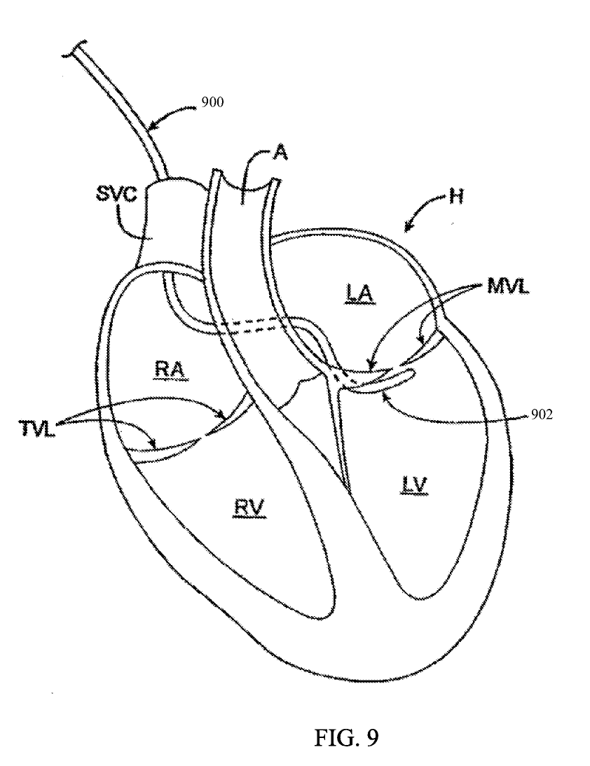

In another variation, a tether may be coupled to an anchor using a back splice. FIGS. 7A-7H illustrate one variation of a method for tying a back splice in a tether. First, the length of the tether segment that will form the back splice is determined. Next, and as shown in FIG. 7A, the tether end is unwound so that filament strands (761), (762), and (763) are spread apart. At least one filament strand is then advanced through the eyelet or other portion of an anchor (not shown). Thereafter, a crown knot is created (FIGS. 7A-7E), and filament strands (761), (762), and (763) are woven back into a portion of the tether that is immediately proximal to the ends of the filament strands. The process is repeated until a desired length of the back splice has been formed. The distal ends of the filament strands may then be cut. FIG. 8 shows an example of a back splice coupled to an anchor.

Although the methods described above show the formation of an eye splice and a back splice from tethers having three filament strands, it should be noted that the general principles described herein may be applied to other variations (e.g., to create an eye splice or a back splice from a tether having three filament strands, four filament strands, five filament strands, ten filament strands, and the like). Moreover, other variations of splices may be used to couple a tether to an anchor. Other non-limiting examples of suitable splice configurations include cut splices, horseshoe splices, long splices, short splices, and side splices. Moreover, in some variations, certain portions of a splice may be tapered to reduce the splice's volume.

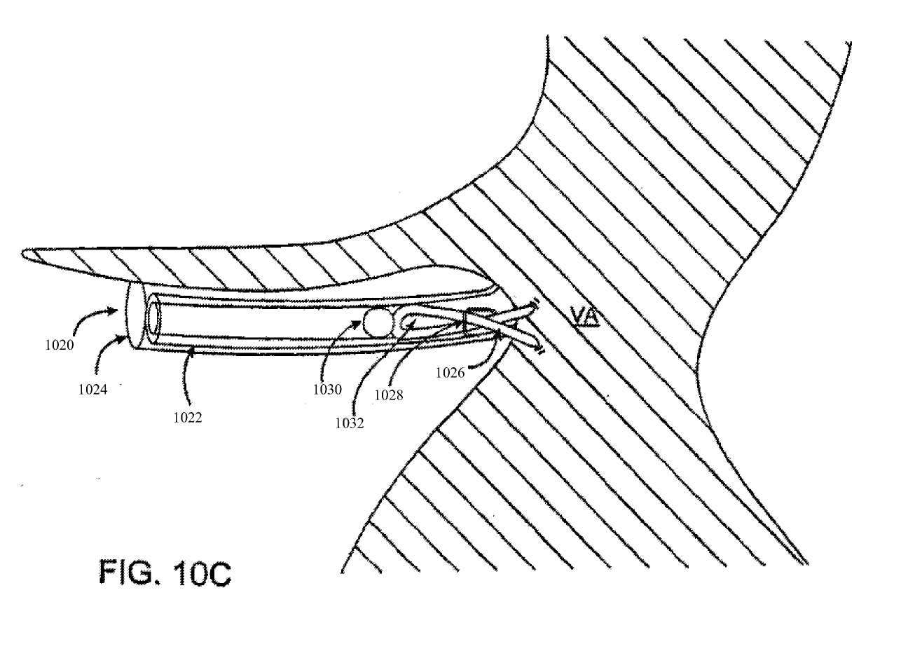

In other variations, a tether and an anchor may be coupled by inserting or otherwise locating a portion of the anchor within an opening or passageway within in the elongate body of the tether. Referring to FIG. 11, for example, anchor (1102) of tether-anchor assembly (1100) may be inserted or placed into an intrabody opening (1106) between strands (1108) of multi-filament or braided tether (1104). Intrabody opening (1106) may be formed by piercing tether (1104) with a sharp instrument or needle, or by axially compressing tether (1104) to cause strands (1108) to bow outward and/or separate from each other. In some variations, the number of strands (1108) to each side of anchor (1102) or intrabody opening (1106) is approximately equal, but in other variations, may be unequal. The total number of strands and the number of strands on each side of the opening may vary. In still other variations, an anchor may be inserted through multiple openings formed in the tether. In the example depicted in FIG. 11, tether-anchor assembly (1100) is configured with two strands (1108) to each side of intrabody opening (1106), but in other examples, the tether may comprise anywhere from about two or three total strands to about ten or twelve total strands (or more), or about four strands to about six or eight total strands.

In some variations, tether (1104) may be chemically-treated or heat-treated, coated and/or infused with an adhesive or other agent, for example, to cause at least some adherence, melting or fusion of strands (1108) to each other. In some examples, one or more of these treatments may resist fraying, dissection or separation of strands (1108). The adhered or fused strand region may be used as a stop structure to resist pullout or separation of anchor (1102) when tether (1104) is tensioned. The region of tether (1104) undergoing treatment may vary, and may include the segment (1110) distal to intrabody opening (1106), the segment (1112) about intrabody opening (1106) and/or the segment (1114) proximal to opening (1106). Segments (1110, 1112 and/or 1114) may have a length in the range of about 1 mm to about 10 mm or more, in other configurations about 2 mm to about 6 mm, and still other configurations about 4 mm to about 8 mm. In some variations comprising heat-setting, tether (1104) may be directly heated by a heat source, but in other examples, a sleeve or tube having a higher melting temperature than tether (1104) may be placed over tether (1104) to control the heat-setting. In one example, the tether may comprise a polyethylene (e.g. ultra-high molecular weight polyethylene) and a fluorinated ethylene propylene tube may be temporarily placed over the tether region during heat treatment. In still other variations, a sleeve or tube may be heat-shrunk or crimped to one or more portions of the tether and/or anchor, which may also be used or function as a stop structure.

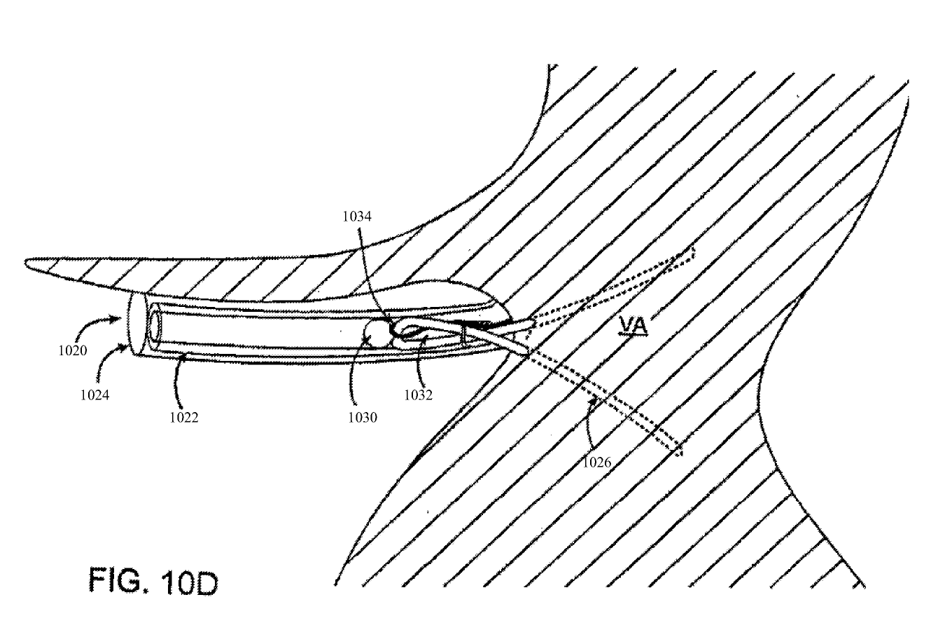

Although the variation described above utilizes a multi-filament tether, in other examples, a monofilament tether may also be used. In FIG. 12, for example, the tether-anchor assembly (1120) comprises an anchor (1122) located in an intrabody opening (1126) of a mono-filament tether (1124). Intrabody opening (1126) may be formed by cutting, piercing or melting the tether material, for example. The intrabody opening may comprise any of a variety of configurations, such as the double-tapered shape of opening (1126), but in other variations, may be oval, circular or polygonal, for example.

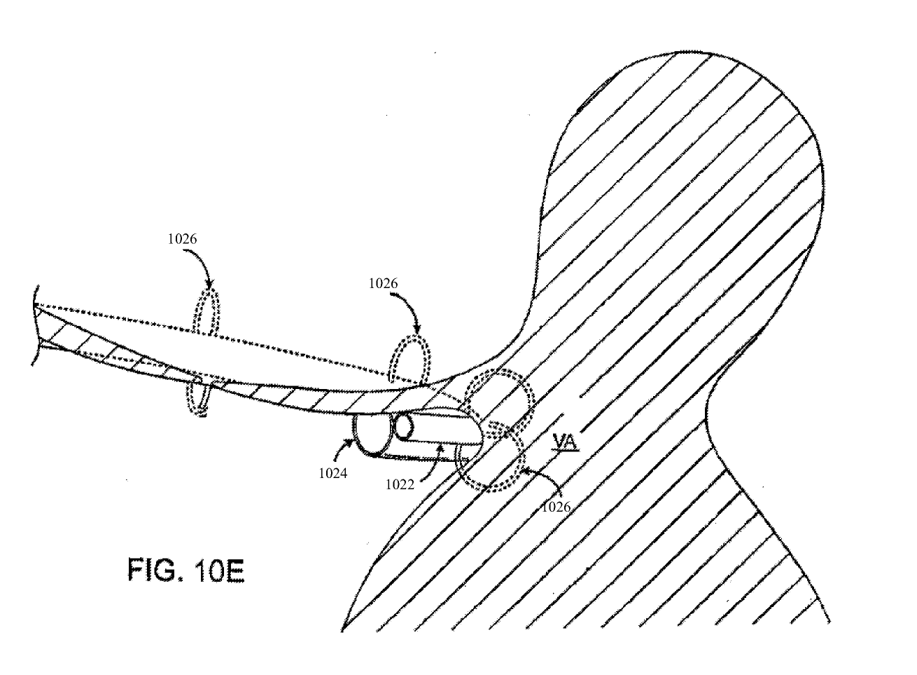

In still other variations the opening(s) of the tether-anchor assembly may be provided in a separate structure attached to the tether. For example, in FIG. 13, tether-anchor assembly (1140) comprises anchor (1142) coupled to tether (1144) using eyelet structure (1148) having anchor opening (1146). Eyelet structure (1148) may be attached to tether (1144) by any of a variety of processes, including but not limited to gluing, melting, or crimping, for example. Eyelet structure (1148) may comprise the same or different material as tether (1144) or anchor (1142), and may comprise a metal and/or non-metal material.

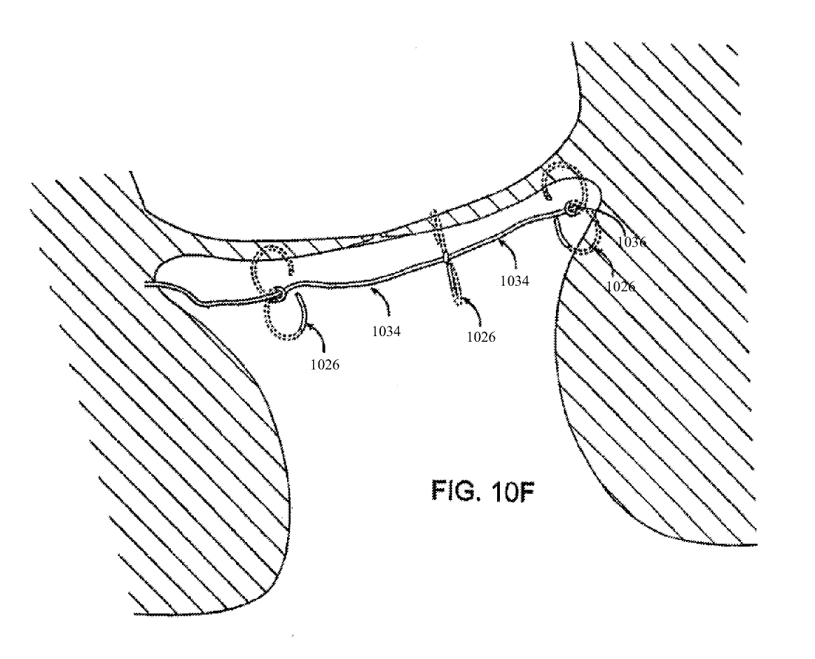

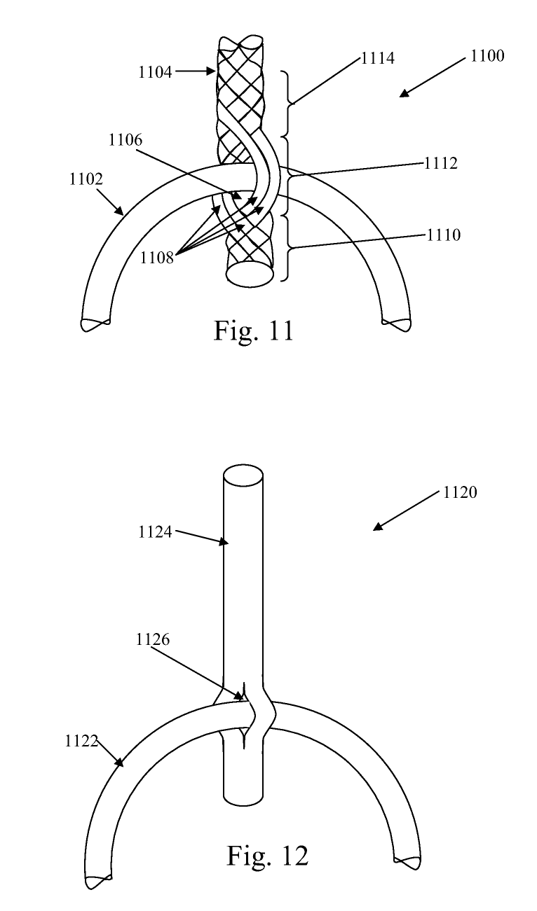

In some variations, the anchor of the tether-anchor assembly may be configured to resist or limit sliding or other movement with respect to the tether. In FIG. 14, for example, the tether-anchor assembly (1160) comprises anchor (1162) coupled to tether (1164) with attached eyelet structure (1168). Opening (1166) of eyelet structure (1168) has a circular cross-sectional shape which restricts relative movement of anchor (1162) beyond limit structures (1170) projecting from anchor (1162). Limit structure (1170) may comprise any of a variety of shapes or configurations and may be flexible, semi-rigid or rigid. In some variations, limit structure (1170) may be angled to facilitate passage through opening (1166) in one direction but to resist passage through opening (1166) in the opposite direction. In still other variations, the opening may be configured with a cross-sectional shape that permits passage of the anchor and its limit structure at a particular rotational or angular orientation of the anchor but to resist passage at a different rotational or angular orientation, e.g. a keyhole shape. In still another variation, illustrated in FIG. 15, tether-anchor assembly (1200) comprises anchor (1202) with narrow segment (1210) coupled to opening (1206) of eyelet structure (1208) that is attached to tether (1204). The coupling may be a fixed coupling, or a slidable and/or rotatable coupling. Opening (1206) is sized or otherwise configured to permit sliding along the exposed length of narrow segment (1210) but movement along the thick segments (1212) of anchor (1202) is restricted.

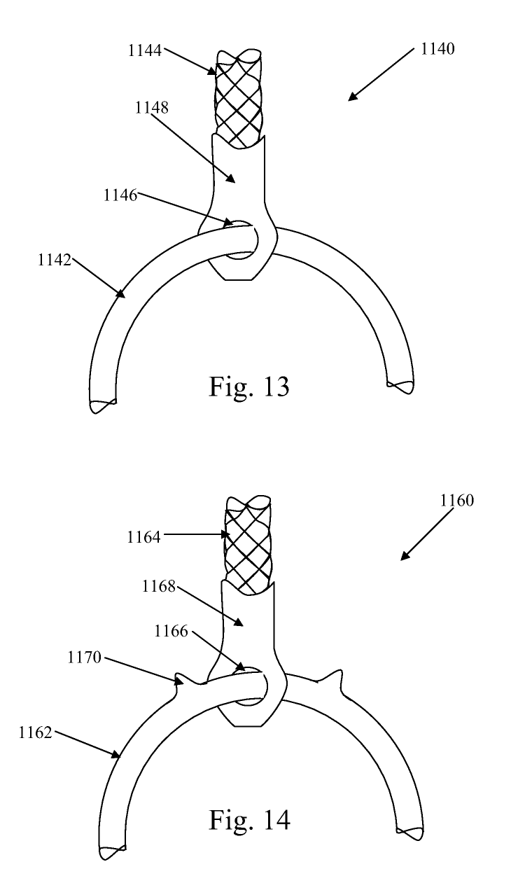

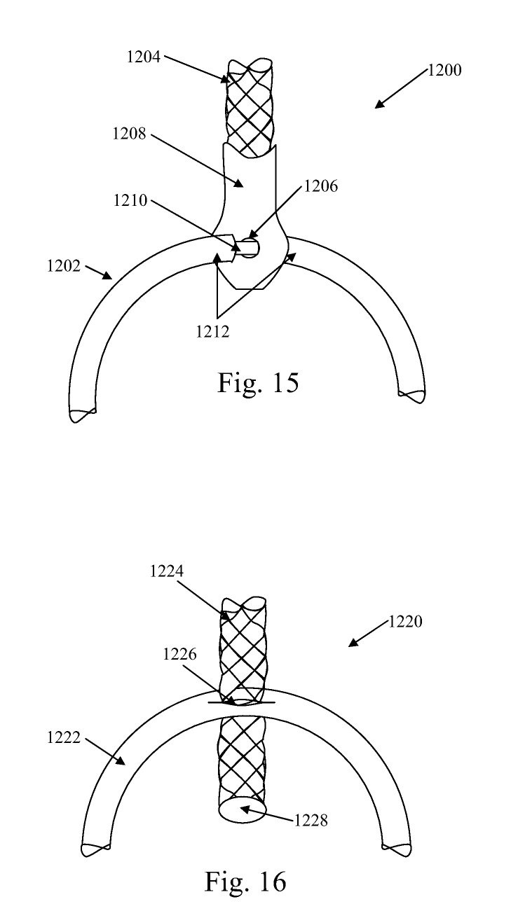

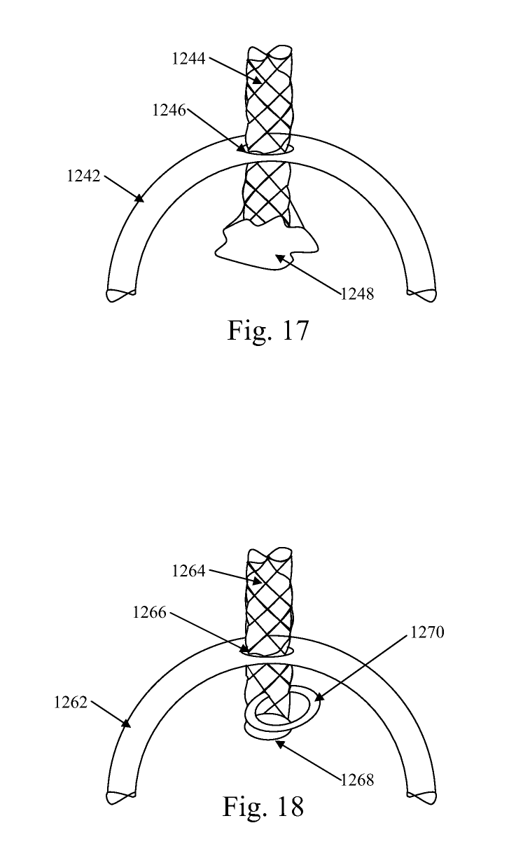

In another variation of tether-anchor assembly (1220) depicted in FIG. 16, the anchor (1222) may comprise an opening (1226) through which the tether (1224) is inserted. Anchor (1222) may be crimped to inserted tether (1224) to resist separation. The distal end (1228) of tether (1224) may also be heated, melted or otherwise enlarged to optionally provide mechanical interference to separation. In an alternate variation illustrated in FIG. 17, the tether (1244) is inserted into an opening (1246) of the anchor (1242) but is not crimped together but the distal end (1246) or region is fused or melted to an enlarged configuration to resist separation. In still another alternate variation depicted in FIG. 18, rather than melting the distal end (1268) of the tether (1264), an interference structure, such as a ring (1270), is inserted through the strands of tether (1264) or otherwise attached to tether (1264) to resist pullout from the opening (1266) of the anchor (1262). In other variations, the interference structure need not have a ring-like shape and may have any of a variety of configurations where at least one cross-sectional dimension of the interference structure is at least larger than the corresponding cross-sectional dimension of the opening in the anchor.

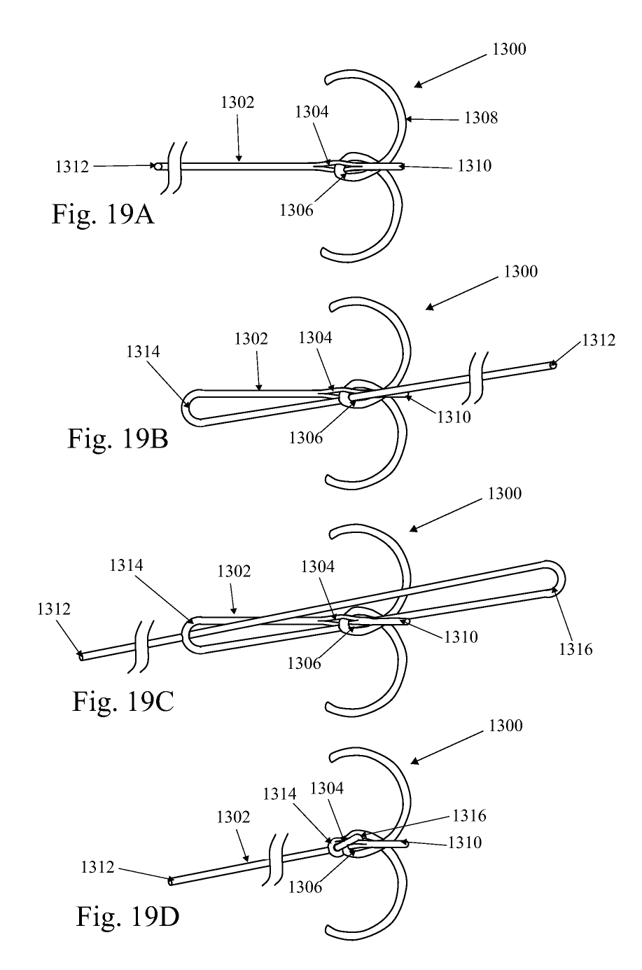

In some variations, the exemplary tether-anchor assemblies described herein, such as those depicted in FIGS. 11 to 18, may be configured by also tying or knotting the tether to the anchor. In some instances, further tying or knotting of a tether initially attached to an anchor without knots may further secure the tether to the anchor, may provide additional frictional resistance to sliding between the tether and the anchor, and/or may enlarge the tether-anchor interface such that tether movement is restricted by the shape or configuration of the eyelet. FIGS. 19A to 19D schematically depict one variation of a tether-anchor assembly (1300) comprising a tether (1302) with an opening (1304) in which the eyelet (1306) or other attachment region of an anchor (1308) is positioned. The distal region (1310) of tether (1302) may be bonded, crimped, chemically-treated, heat-fused or otherwise configured to resist separation of anchor (1308) from opening (1304) of tether (1302). Although tether (1302) is schematically depicted in FIGS. 19A to 19D without individual filaments to simplify the illustration of the exemplary knotting procedure, tether (1302) may be a multi-filament or a monofilament tether.

In this exemplary variation, distal region (1310) of tether (1302), if any, is positioned to one side of anchor (1308), e.g. the “top” or visible side of anchor (1308), but in other variations may be positioned on the bottom side of anchor (1308). As illustrated in FIG. 19B, the proximal end (1312) of tether (1302) is passed through eyelet (1306) from the bottom side of anchor (1308) (or the opposite side from where fused distal region (1310) is positioned) to form a first or proximal loop (1314). As shown in FIG. 19C, a second or distal loop (1316) may then be formed by passing proximal end (1312) of tether (1302) through first loop (1314) from a top to bottom direction (or from same to the opposite side of loop (1314) with respect to fused distal region (1310). Fused distal region (1310) may be positioned within second loop (1316), as shown in FIG. 19C, but in other variations may be positioned outside of second loop (1316). The positioning may occur during or after second loop (1316) is formed. As illustrated in FIG. 19D, tether (1302) may then be tightened or cinched to close first and second loops (1314 and 1316) around eyelet (1306) and fused distal region (1310). First and/or second loops (1314 and/or 1316) may also tighten or cinch around opening (1304) of tether (1302), which may or may not further secure and/or restrict tether (1302) to anchor (1308).

Although the example depicted in FIGS. 19A to 19D comprises tether (1302) with opening (1304) and distal region (1310), the depicted knot procedure may also be performed with tethers lacking a distal region. Also, the knot procedure(s) that may be used with tether-anchor assemblies comprising non-knot attachments are not limited to the knot process depicted in FIGS. 19A to 19D. Other types of knots that may be used to enclose a portion of the anchor include the other knots described herein for tethers solely attached to anchors using knots. These knots may or may not loop around a distal region of the tether, if any, and may or may not use the distal region, if any, to form the knot itself, e.g. an overhand knot. In some further variations, the one or more knots formed may also be adhered together using heat, chemicals, and/or an adhesive.

In some variations, the tether-anchor configurations described here may be used to bind tissue to other tissue and/or to bone. In certain variations, the tether-anchor assemblies described here may be used to attach a graft or other material foreign to the body to tissue and/or bone in the body.

Anchors for use with the methods and devices described here may be any suitable anchor. The anchors may be made of any suitable material, may be any suitable size, and may be of any suitable shape. The anchors may be made of one material or more than one material. Examples of anchor materials include any suitable biocompatible materials, such as super-elastic or shape memory materials, such as nickel-titanium alloys (e.g., nitinol) and spring stainless steel. Additional examples of materials include metals (e.g., titanium), polymers (e.g., polyester, nylon, polylactic acid, polyglycolic acid), and combinations thereof. An anchor may be made of a single material, or it may be made of multiple materials. In some variations, an anchor may be made of or formed from a single piece of material. For example, a linear material may be formed into an anchor. In certain variations, an anchor may be cut or etched from a sheet of material. In some variations, an anchor may include different regions that are connected or joined together. These different regions may be made of the same material, or they may be made of different materials. The different regions may include regions having different physical or material properties, such as material strength, flexibility, ductability, elasticity, and the like. For example, an anchor may have an eyelet region comprising a material having a different (e.g., a decreased or increased) stiffness compared to one or more leg regions of the anchor. As previously described, the eyelet region may also have surface characteristics that are different from other regions of the anchor.

In some variations, an anchor and/or tether may be made of (or may contain a region or coating of) a biodegradable or bioabsorbable material in addition to, or as an alternative to, the biocompatible materials described above. Biodegradable or bioabsorbable portions of the anchor may allow time-controlled changes in the mechanical or biochemical properties of the anchor and in the interaction of the anchor with tissue. For example, an anchor may comprise an outer layer that dissolves over time, rendering the anchor thinner and more flexible. Thus, an anchor may initially be quite thick (e.g., providing an initial strength or stiffness), but after insertion into tissue, the outer layer may dissolve or be removed, leaving the anchor more flexible, so that it can better match the tissue compliance. In some variations, the anchors and/or the tether are formed entirely of one or more biodegradable and/or bioabsorbable materials, such that the tether-anchor assembly (or some component thereof) can degrade and/or be absorbed over time. The time for biodegradation, for example, may be timed to correspond to fibrous tissue growth into and/or around the tether-anchor assembly. In this way, once sufficient fibrous tissue growth has encapsulated or surrounded the tether-anchor assembly so as to render the assembly useless, the assembly (or a component thereof) may degrade. In certain variations, a tether-anchor assembly may be configured to encourage or promote growth of new tissue (including fibrous scar tissue) around the assembly and/or into the assembly. Methods and materials for promoting fibrous tissue growth and for forming new annular bands of tissue are described, for example, in U.S. patent application Ser. No. 11/255,400, which is hereby incorporated by reference in its entirety.

Any of the anchors and/or tethers described here may be treated or coated with any suitable material in any appropriate manner. For example, some variations of anchors may be treated with a therapeutic substance (e.g., an anti-inflammatory substance, an anticoagulant or thromboresistant substance, an antiproliferative substance, a pro-proliferative substance, etc.) to promote healing. Certain variations of anchors may be coated with a Vascular Endothelial Growth Factor (VegF), Fibroblast Growth Factor (FGF), Platelet-Derived Growth Factor (PDGF), Transforming Growth Factor Beta (TGFbeta, or analogs), insulin, insulin-like growth factors, estrogens, heparin, and/or Granulocyte Colony-Stimulating Factor (G-CSF). The anchors may also be coated with one or more materials or agents to promote adhesion, and/or may comprise one or more materials that enhance the anchors' visibility during diagnostic procedures. For example, an anchor may comprise one or more radiopaque materials (e.g., a metal such as gold or aluminum), or other contrast-enhancing agents. The agent selected may depend upon the material from which the anchor is made, and the imaging modality used.

Other examples of anchor and/or tether coating materials include lubricious coating materials. For example, in certain variations, an anchor may comprise an eyelet region (or other suitable tether attachment point), or a portion thereof, which includes a lubricious coating. The lubricious coating may promote knot tie-down performance (i.e., the ease of tying a knot onto the anchor). As an example, the lubricious coating may cause a tether to slide over and/or through different regions of the anchor more easily during knot formation, such that a knot may be formed in the tether relatively quickly. The lubricious coating may alternatively or additionally decrease abrasion between the anchor and a tether, which may help to maintain the tether's structural integrity. Lubricious coatings may alternatively or additionally be used for one or more other purposes. It should also be noted that in some instances, it may not be desirable to have a lubricious coating on one or more (or all) regions of an anchor, such as an eyelet region.

Some variations of lubricious coating materials may be hydrophilic, while other variations of lubricious coating materials may be hydrophobic. For example, a hydrophobic polymer, such as a polyxylene polymer (e.g., parylene) may be used. Additional examples of suitable lubricious coating materials include polytetrafluoroethylene (PTFE) and wear-resistant deposition coatings including but not limited to ceramics such as nitride-bonded silicon carbide (e.g., Nitron™). Other lubricious coating materials may also be used, as appropriate.

In some variations, an eyelet or another anchor and/or tether region, or a portion thereof, may be coated with one or more materials which may promote the security of the coupling between the anchor and a tether. For example, the eyelet or other anchor region may be coated with a material that increases the coefficient of friction of the anchor in that region. As a result, the friction between the anchor region and a tether contacting the anchor region may be enhanced. This enhanced friction may result in a more secure coupling between the anchor and the tether.

In certain variations, an eyelet or another anchor and/or tether region, or a portion thereof, may be coated with one or more materials that are lubricious during implantation of the anchor, but that later become less lubricious. As the material or materials become less lubricious, they may provide enhanced security of the coupling between the anchor and a tether. For example, a region of the anchor may be coated with a volatile material that, as it volatilizes (e.g., after implantation of the anchor at a target site), solidifies and provides enhanced engagement with the tether.

Coatings may be used to elute one or more drugs, as described above. For example, an outer layer may comprise a drug (or other dissolvable or removable layer) that exposes another layer (e.g., another drug layer) after it dissolves or is removed. Thus, an anchor and/or tether may deliver more than one drug in a controlled fashion. The release of a drug (or drug coating) may be affected by the geometry of the anchor, or the way in which the drug is arranged on or within the anchor. Of course, the anchors and/or tether may comprise one or more drug depots or reservoirs for release of one or more agents or substances, and these depots or reservoirs may have any suitable geometry or configuration (for example, pits, slots, bumps, holes, crevices, grooves, recesses, etc.). Such anchor modifications may also allow for tissue ingrowth. Of course, the anchor and/or tether may also be impregnated with or extruded with one or more agents or substances.

It is also contemplated that different regions of an anchor and/or tether may comprise different coatings. As an example, one region of an anchor may be coated with a lubricious coating material, while another region of the anchor may be coated with a drug-eluting coating material that is different from the lubricious coating material. Anchors or portions of anchors may be coated with just one layer of a coating material, or multiple layers of different coating materials.

Some variations of anchors may undergo one or more surface treatments, such as electropolishing, texturing (e.g., by ion beam etching, photoetching, etc.), tempering (e.g., thermal or photo tempering), and the like. Additional examples of appropriate surface treatments may include chemical etching, grit or bead blasting, or tumbling in abrasive or polishing media.

Certain variations of anchors may be sterilized. For example, an anchor may be irradiated, heated, or otherwise treated to sterilize the anchor. Sterilized anchors may be packaged to preserve sterility.

Examples of suitable anchor shapes or configurations include T-tags, rivets, staples, hooks (e.g., C-shaped or semicircular hooks, curved hooks of other shapes, straight hooks, barbed hooks), multiple looped anchors, tacks, screws, and clips. The anchors may have one or more eyelets or eyelet regions, may define one or more lumens, may have one or more apertures therethrough, or may not. The anchors may be configured to self-expand and self-secure into tissue, but need not be configured in such a fashion. Multiple anchors of the same shape may be used, or multiple anchors having different shapes may be used. Similarly, multiple anchors of the same size may be used, or multiple anchors having different sizes may be used. Illustrative examples of suitable anchors are described in more detail, for example, in U.S. patent application Ser. Nos. 11/202,474; 11/894,340; 11/894,368; 11/894,397; 11/894,463; and 11/894,468, all of which are hereby incorporated by reference in their entireties. Moreover, while anchors have been described, any other types of suitable fasteners or implants may be used, as appropriate. Additionally, some procedures employing the devices and methods described herein may not involve any anchors or other types of fasteners, for example, they may involve tether-implant assemblies.

In some variations, an anchor such as anchor (180) (FIG. 1A) may be used. Of course, any of the anchors used may have any suitable cross-sectional shape, such as one that is circular, oval, triangular, quadrilateral (e.g., rectangular, square, trapezoidal), pentagonal, hexagonal, octagonal, for example. The cross-sectional dimensions (e.g., thickness, width, diameter) and/or shape of the anchors may be uniform, but need not. For example, in some variations, the eyelet region of the anchor is of a greater or lesser thickness than the rest of the anchor.

Certain variations of anchors may include one or more tissue-engaging features. These features may help to secure the anchors to tissue, implants, or grafts. For example, the anchors may include features that increase friction on the anchors' surfaces, capture tissue, restrict anchor movement, and/or limit the likelihood of pullout of the anchors. As an example, an anchor may have ends comprising one or more barbs and/or hooks. In some variations, regions other than the ends of anchor legs (e.g., the body of the legs or the eyelet region) may also include barbs and/or hooks for gripping.

Thus, an anchor may include regions having increased coefficients of friction. In addition to the barbs described above, or as an alternative to the barbs, an anchor may include tines, pores, holes, cut-outs, and/or kinks. These features may increase friction and resistance to pullout, and (as described above) may also allow for tissue ingrowth, which may help prevent accidental dislodgement of the anchor after it has been delivered. The surface of an anchor may also be coated or textured to reduce friction or to increase interaction between the anchor and tissue, implants, or other material. In some variations, the surface of an anchor eyelet region may include ridges and/or notches to create unevenness and thereby increase the surface's coefficient of friction.

In certain variations, and as described above, an anchor may include an eyelet or opening through which a tether passes. However, in other variations, an anchor may not include an eyelet, but may be secured to a tether by coupling the tether to a portion of the anchor (e.g., an anchor member). Additionally, in some variations, an anchor may include an eyelet, but a tether may be coupled to a different portion of the anchor, rather than the eyelet.

In certain variations of anchors that include an eyelet, the size and shape of the eyelet may be defined by an eyelet region that is part of the anchor. In some variations, the eyelet may be closed (i.e., the eyelet may be substantially enclosed by an eyelet region), but in other variations, the eyelet may be partially open (i.e., the eyelet may not be completely enclosed by an eyelet region). The eyelet and/or eyelet region may be of any appropriate shape or size at any location, and may also change shape and/or size based on the configuration of the anchor. For example, in some variations, when the anchor is in a deployed configuration, the eyelet and/or eyelet region may be larger (e.g., wider) than when the anchor is in a delivery configuration. As another example, in certain variations, the eyelet and/or eyelet region may be more elliptical when the anchor is in a delivery configuration, and more rounded when the anchor is in a deployed configuration. In other variations, the size and shape of the eyelet and/or eyelet region may remain substantially constant regardless of the anchor's configuration. In some variations, the eyelet region may comprise one or more structural features, such as ridges, notches, apertures, and the like. These structural features may promote the tether-anchor assembly's knot security.

An anchor may have any suitable number of legs. Although anchor (180) of FIG. 1A comprises two legs (181) and (182), some variations of anchors may comprise only one leg, or may comprise three legs, four legs, five legs, ten legs, etc. It should be noted that the anchor illustrated in FIG. 1A is merely exemplary and that any of a variety of anchors may be used in the tether-anchor assembly as described herein throughout.

The anchors described herein may be deployed in any tissue or bone, or into one or more implants or materials that are configured for attachment to tissue or bone, etc. For example, the anchors may be inserted into heart tissue, such as the sinoatrial node, the atrioventricular node, Perkinje fibers, myocardium, etc. The anchors may also be used to treat or repair patent foramen ovale (PFO), obesity (e.g., insertion into the stomach, the GI, the GI/GE junction), bowel anastamosis, appendectomy, rectal prolapse, hernia repair, uterine prolapse, bladder repair, tendon end ligament repair, joint capsulary repair, attachment of soft tissues to bone, nerve repair, etc. The anchors may also be used to attach implants or grafts. For example, an anchor may be used to attach annuloplasty rings or valves to an annulus. Additionally, the anchors described herein may be used to close vascular access ports for percutaneous procedures.

Other exemplary anchors for use with the devices, methods, and kits described herein include those described in U.S. patent application Ser. No. 11/202,474 entitled, “Devices and Methods for Anchoring Tissue,” filed on Aug. 11, 2005, which is hereby incorporated by reference in its entirety.

As mentioned briefly above, the tethers contemplated for use with the tether-anchor assemblies described here may be comprised of any of a variety of materials. In some variations, a tether may be formed of a biodegradable material, which may be configured to degrade over a period of days, weeks, months, or even years. Examples of suitable biodegradable materials include, but are not limited to, polyglactin (e.g., Vicryl™, Polyglactin 910™), polydioxanone (e.g., PDSm™), polyglecaprone 25 (e.g., Monocryl™), polyglyconate (e.g., Maxon™), polyglycolic acid (e.g., Dexon™), polylactic acid, and processed collagen (e.g., catgut). The tethers, or a portion thereof, may also be formed of one or more non-biodegradable materials. Examples of non-biodegradable materials include, but are not limited to, polyester (e.g., Dacron™, Ethibond™, Ethiflex™, Mersilene™, Ticron™), polypropylene (e.g., Prolene™, Surgilene™), nylon (e.g., Ethilon™, Dermalon™), polytetrafluoroethylene, metal wire (e.g., steel, copper, silver, aluminum, various alloys), silk, linen, and GORE-TEX®. In some variations, a tether may be formed of a combination of one or more biodegradable materials and one or more non-biodegradable materials.

Tethers may be monofilament tethers (comprising a single strand of material) or may be multifilament tethers (comprising a plurality of strands of material braided together). In some instances, monofilament tethers may require more ties or throws to achieve adequate knot security, as compared to equivalent braided multifilament tethers. Thus, the resulting knot may have a larger volume and occupy more space. Accordingly, in some instances, the type of tether that is selected for a procedure may affect the total knot volume.

Some variations of multifilament tethers may provide better tying characteristics than monofilament tethers. For example, multifilament tethers may exhibit greater flexibility and/or higher coefficients of friction than monofilament tethers. A higher coefficient of friction may provide a multifilament tether with the capability of achieving greater knot security than a monofilament tether. As a result, certain variations of multifilament tethers may achieve a given level of knot security using fewer ties or throws than a monofilament tether.

In some variations, a tether may not be coated, but in other variations, all of part of a tether may be coated. For example, a tether may be coated with at least one coating to improve certain tether characteristics, such as lubricity, knot security, anti-infective or anti-bacterial properties, and/or abrasion resistance. Non-limiting examples of coatings include wax (e.g., beeswax, petroleum wax, polyethylene wax), silicone (e.g., Dow Corning silicone fluid 202A), silicone rubbers (e.g., Nusil Med 2245, Nusil Med 2174 with a bonding catalyst), PTFE (e.g., Teflon, Hostaflon), PBA (polybutylate acid), and ethyl cellulose (Filodel), silver, fibrin glue, Polymethylmethacrylate (PMMA) Cement, Hydroxyapatite Cement, antibiotic spray, collagens, liposomes, collagen scaffold, polylactic acid, Polyhydroxyethyl methacrylate (pHEMA), Polyvinylalcohol and gum arabica blend matrix, antibiotics, and the like. A single coating material may be used, or combinations of coating materials may be used. In certain variations, a tether may alternatively or additionally be coated with one or more beneficial or therapeutic agents.

Of course, as described above, the anchor or any portion thereof may also be coated with any of the above-mentioned compounds. Coatings may be placed on the anchor body and/or the tether using any suitable technique, e.g., plasma deposition, dipping, spraying, and wiping, for example.

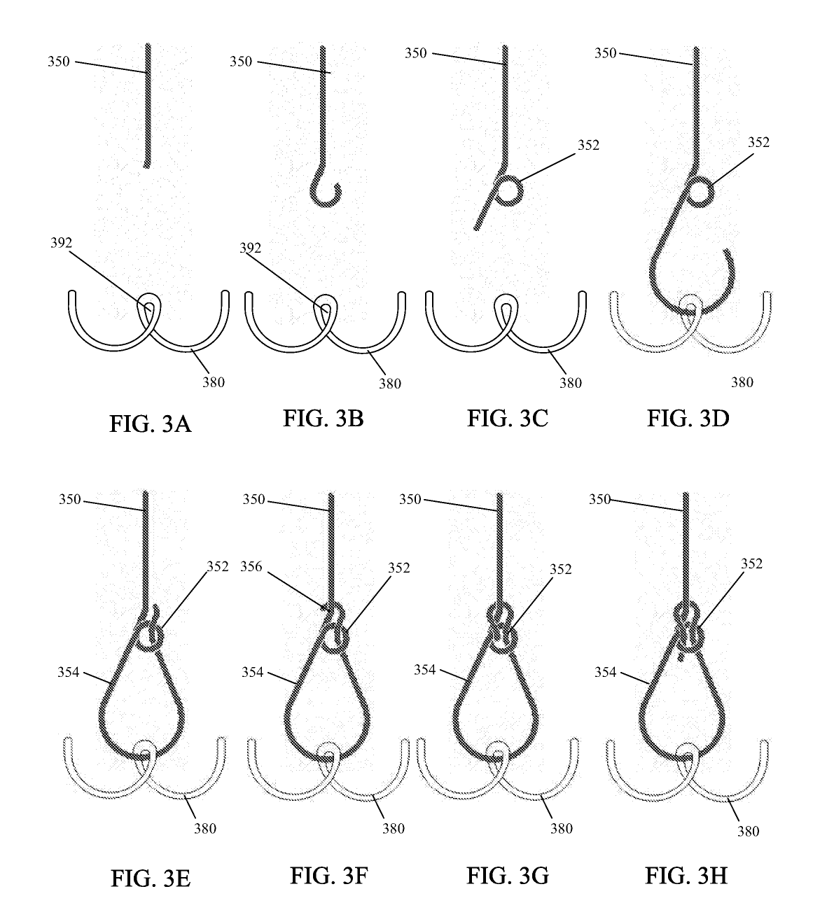

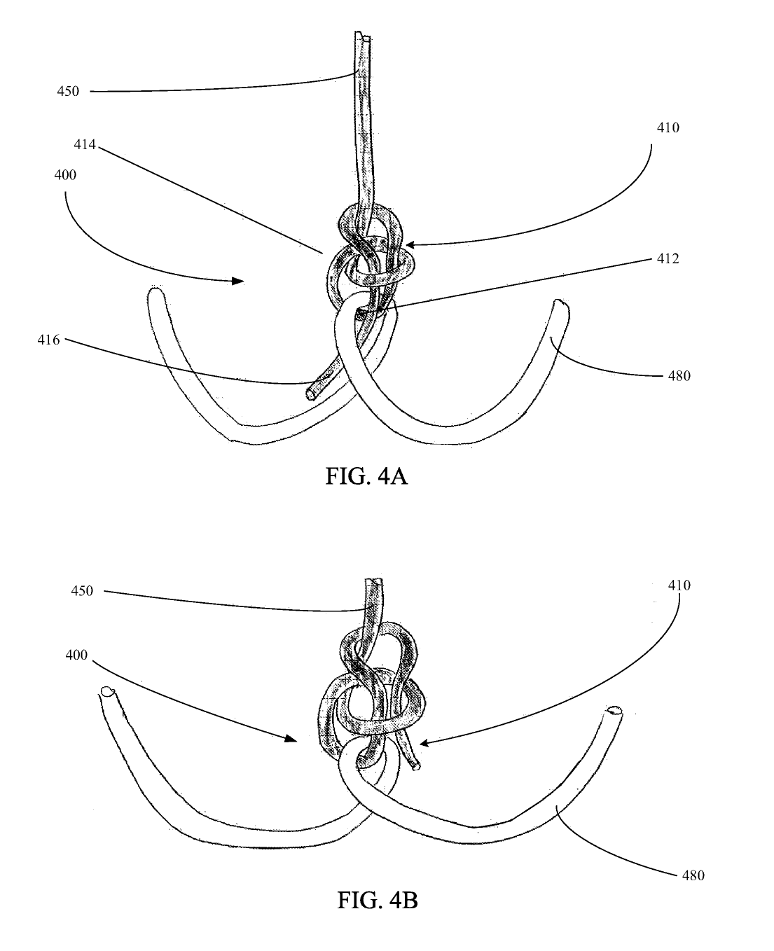

In some variations, knot security may be enhanced by using a braided tether, because of the braided tether's irregular surface. In certain variations, knot security may be enhanced by using a tether that, while not braided, has a surface with certain structural characteristics that contribute to the formation of a secure knot. For example, FIG. 2A shows a variation of a tether (250) comprising notches (252), and FIG. 2B shows another variation of tether (250) comprising ridges (254). It is contemplated that notches (252) and/or ridges (254) may have any suitable cross-sectional shape (e.g., round, oval, square, triangular, rectangular, polygonal) and any suitable height or depth. Texturing the surface of a tether may increase the coefficient of friction of the tether and may promote knot security and/or knot strength. In certain variations, a tether may comprise a textured region that is used to form a knot or splice, and may also comprise one or more other regions that are not textured.

Tether size may generally be measured by the tether's diameter. When the tether is made of a standard suture material, it is typically designated with a number ranging from 5 (e.g., a heavy braided tether for orthopedics having a diameter range of from about 0.7 mm to about 0.79 mm) to a 0 (e.g., a fine monofilament tether for ophthalmics having a diameter range of from about 0.01 mm to about 0.019 mm), under United States Pharmacopeia (USP) standards. As tether size increases, tether tensile strength and knot security may also increase. However, in some instances, an increase in tether size may also correspond to an increase in knot volume.

It is contemplated that tether size selection may be dependent in part on the particular procedure to be performed. In some variations, a tether may have a diameter of from about 0.01 mm to about 0.8 mm, sometimes from about 0.03 mm to about 0.5 mm, and sometimes from about 0.1 mm to about 0.25 mm. Tether selection may also be dependent in part on various anchor dimensions (e.g., the shape and/or cross-sectional area of the eyelet, the thickness of the eyelet region, etc.). For example, in some variations, a tether may have a cross-sectional area that is from about 0.01% to about 99% of the eyelet (or other tether attachment region) cross-sectional area, sometimes from about 1% to about 10%, and other times from about 15% to about 33%. In certain variations, a tether may have a diameter that is from about 0.1% to about 300% of the eyelet (or other tether attachment region), sometimes from about 1% to about 100%, and sometimes from about 10% to about 80%.

In addition to material, dimensions, structure, and configuration, other considerations involved in tether selection may include breaking strength (i.e., the limit of tensile strength at which tether failure occurs), capillarity (i.e., the extent to which absorbed fluid is transferred along the tether), and flexibility (i.e., the ability of the tether to regain its original form and length after deformation).

In some variations, one or more regions of a tether may be further treated to augment the characteristics of the tether. For example, one or more segments of the tether may be heat-treated or chemically treated to fuse, melt or bond one region of a tether to another region, to fuse, melt or bond one tether to another tether, and/or to melt or bond the strands of a multi-filament tether together. In some variations, heat or chemical bonding may be used to resist separation of the bonded materials, but in other variations, may be used to increase or decrease the smoothness of the tether surface, or to increase or decrease the stiffness or flexibility of the tether. One of skill in the art will understand that the availability of heat or chemical melting of the tether and the effect on the tether will vary depending upon the particular tether material.

In some variations, one or more regions of the tether and/or anchor may be coated or infused with one or more other materials to augment the characteristics of the tether-anchor assembly. For example, the tether may be coated with a lubricious substance, anti-thrombogenic material, or an anti-proliferative agent. Application of the additional materials may be performed using any of a variety of processes, including but not limited to spray coating, dip coating or soaking. In some variations, excess material applied to the tether and/or anchor may be removed using an airjet. In still other variations, the applied materials may be further treated by heat or light (e.g. UV), for example, to cure the materials.

### EXAMPLES

The following examples describe the use of anchors for treating a cardiac valve tissue. These examples are only intended to illustrate one possible use of the anchors, anchor delivery devices, and anchor systems, and should not be considered limiting.

When used for treatment of a cardiac valve, the methods described herein may involve contacting an anchor delivery device with valve annular tissue, delivering a plurality of anchors from one or more anchor delivery devices, where the anchors are coupled to a tether, and drawing the anchors together to tighten the annular tissue. Devices may include an elongate catheter having a housing at or near the distal end for releasably housing a plurality of coupled anchors, as well as delivery devices for facilitating advancement and/or positioning of an anchor delivery device. Devices may be positioned such that the housing abuts or is close to the annular tissue, such as in a location within the left ventricle defined by the left ventricular wall, a mitral valve leaflet and chordae tendineae. Self-securing anchors having any of a number of different configurations may be used in some variations. Additional devices include delivery devices for facilitating delivery and/or placement of an anchor delivery device at a treatment site.

Certain variations of methods described herein may be performed on a beating heart. Access to the beating heart may be accomplished by any available technique, including intravascular, transthoracic, and the like. In addition to beating heart access, some variations of methods described herein may be used for intravascular stopped heart access as well as stopped heart open chest procedures.

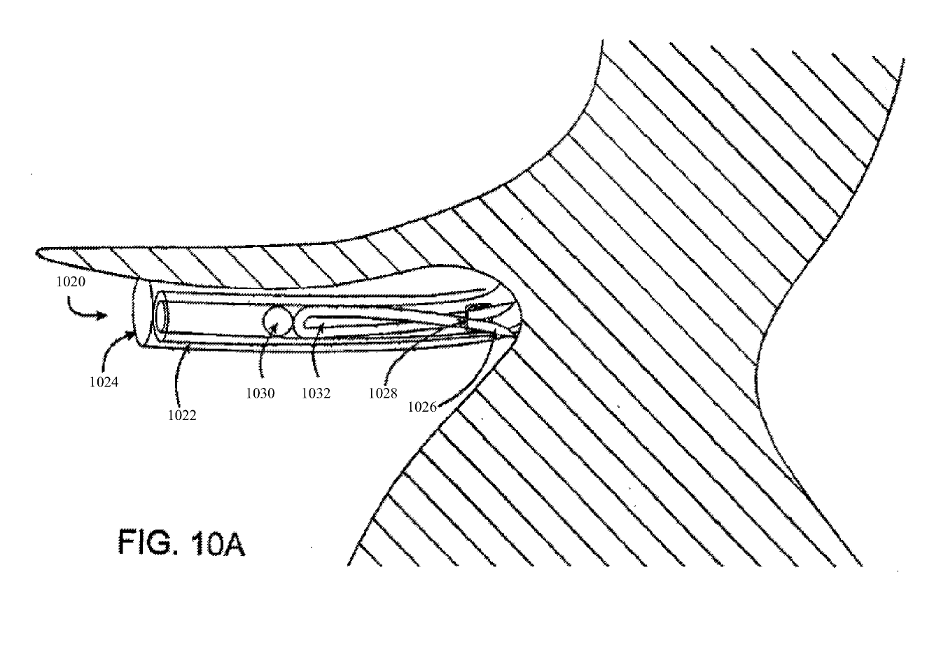

Referring now to FIG. 9, a heart (H) is shown in cross section, with an elongate anchor delivery device (900) introduced within heart (H). Anchors may be delivered or inserted into tissue (including heart tissue, as described below) using any appropriate delivery device. In the example shown in FIG. 9, delivery device (900) comprises an elongate body with a distal portion (902) configured to deliver anchors to a heart valve annulus. In some variations, the elongate body may comprise a rigid shaft, while in other variations it may comprise a flexible catheter, so that distal portion (902) may be positioned in heart (H) and under one or more valve leaflets to engage a valve annulus via a transvascular approach. Transvascular access may be gained, for example, through the internal jugular vein (not shown) to the superior vena cava (SVC) to the right atrium (RA), across the interatrial septum to the left atrium (LA), and then under one or more mitral valve leaflets (MVL) to a position within the left ventricle (LV) under the valve annulus (not shown). Alternatively, access to the heart may be achieved via the femoral vein and the inferior vena cava. In other variations, access may be gained via the coronary sinus (not shown) and through the atrial wall into the left atrium. In still other variations, access may be achieved via a femoral artery and the aorta, into the left ventricle, and under the mitral valve. It is contemplated that any other suitable access route may also be used.

In other variations, access to heart (H) may be transthoracic, with delivery device (900) being introduced into the heart via an incision or port on the heart wall. Even open heart surgical procedures may benefit from methods and devices described herein. Furthermore, some variations may be used to enhance procedures on the tricuspid valve annulus, adjacent the tricuspid valve leaflets (TVL), or any other cardiac or vascular valve. Therefore, although the following description typically focuses on minimally invasive or less invasive mitral valve repair for treating mitral regurgitation, the knot configurations, tether-anchor assemblies, devices, and methods described herein are in no way limited to that use.

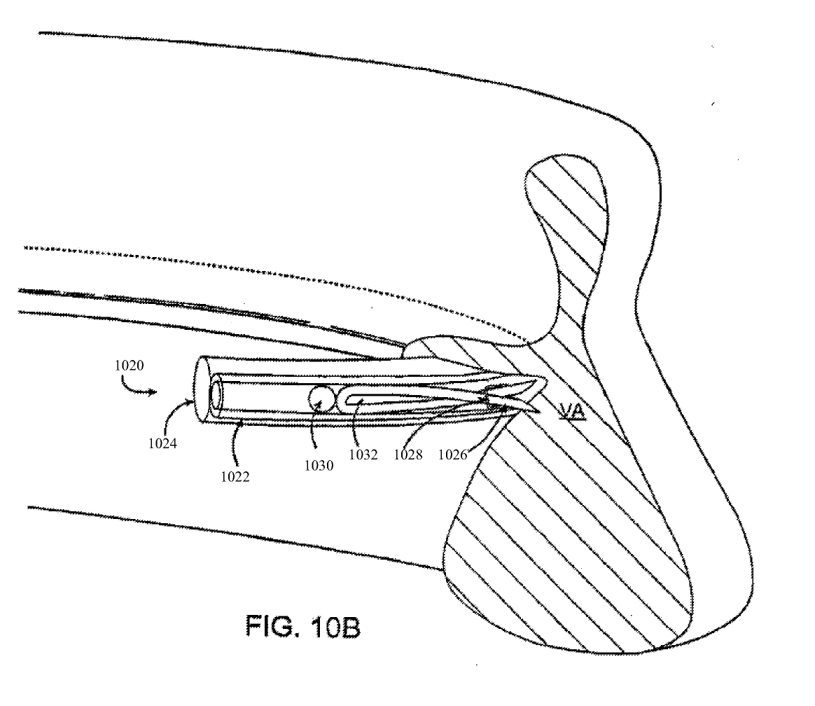

With reference now to FIGS. 10A-10F, a method is shown for applying a plurality of tether-coupled anchors (1026) to a valve annulus or annular tissue (VA) in a heart. As shown in FIG. 10A, an anchor delivery device (1020) is first contacted with the annular tissue such that openings (1028) are oriented to deploy anchors (1026) into the annular tissue. Such orientation may be achieved by any suitable technique. In one variation, for example, a housing (1022) having an elliptical cross-sectional shape may be used to orient openings (1028). Contact between housing (1022) and the annular tissue may be enhanced by expanding expandable member (1024) to wedge housing (1022) within a corner adjacent the annulus.

Generally, delivery device (1020) may be advanced into any suitable location for treating any valve by any suitable advancing or device placement method. Many catheter-based, minimally invasive devices and methods for performing intravascular procedures, for example, are well known, and any such devices and methods, as well as any other devices or methods later developed, may be used to advance or position delivery device (1020) in a desired location. For example, in one variation a steerable guide catheter is first advanced in retrograde fashion through an aorta, typically via access from a femoral artery. The steerable catheter is passed into the left ventricle of the heart and thus into the space formed by the mitral valve leaflets, the left ventricular wall and cordae tendineae of the left ventricle Once in this space, the steerable catheter is easily advanced along a portion (or all) of the circumference of the mitral valve. A sheath is advanced over the steerable catheter within the space below the valve leaflets, and the steerable catheter is removed through the sheath. Anchor delivery device (1020) may then be advanced through the sheath to a desired position within the space, and the sheath may be removed. In some cases, an expandable member coupled to delivery device (1020) may be expanded to wedge or otherwise move delivery device (1020) into the corner formed by the left ventricular wall and the valve leaflets to enhance its contact with the annular tissue. Of course, this is but one exemplary method for advancing delivery device (1020) to a position (e.g., for treating a valve), and any other suitable method, combination of devices, etc. may be used.

As shown in FIG. 10B, when delivery device (1020) is positioned in a desired location for deploying anchors (1026), anchor contacting member (1030) is retracted to contact and apply force to a most-distal anchor to begin deploying the anchor through aperture (1028) and into the annular tissue. FIG. 10C shows the anchor further deployed out of aperture (1028) and into the annular tissue. FIG. 10D shows the annular tissue transparently so that further deployment of anchors (1026) can be seen. As shown, in one variation, anchors (1026) include two legs that move upon release from housing (1022) and upon contacting the annular tissue. Between the two legs, an anchor (1026) may be looped or have any other suitable eyelet or other device for allowing slidable or fixed coupling with a tether (1034).

Referring now to FIG. 10E, one variation of anchors (1026) is shown in a fully deployed or nearly fully deployed shape, with each leg being curved. Of course, anchors (1026) may have any other suitable deployed and undeployed shapes, as described more fully above.

FIG. 10F shows anchors (1026) deployed into the annular tissue and coupled with tether (1034), with the most-distal anchor fixedly coupled to tether (1034) at attachment point (1036) using one or more of the knots and/or splices previously described.

At this stage, tether (1034) may be pulled to cinch the anchors and thereby tighten the annulus, thus reducing valve regurgitation. In some variations, valve function may be monitored by means such as echocardiogram and/or fluoroscopy, and tether (1034) may be pulled, loosened, and/or adjusted to achieve a desired amount of tightening as evident via the employed visualization technique(s). Once a desired amount of tightening has been achieved, tether (1034) may be attached to a most-proximal anchor (or two or more most-proximal anchors) using any suitable technique. Tether (1034) is then severed proximal to the most-proximal anchor, thus leaving the cinched, tether-coupled anchors (1026) in place along the annular tissue. Attachment of tether (1034) to the most-proximal anchor(s) may be achieved via adhesive, knotting (e.g., using one or more of the knots and/or splices described above), crimping, tying or any other suitable technique. Moreover, tether (1034) may be severed using any suitable technique, such as with a cutting member coupled to housing (1022). In some variations, only the most-distal and the most-proximal anchors are fixedly coupled to the tether, while the other anchors are slidably coupled to the tether. In other variations, however, anchors other than the most-distal and the most-proximal anchors may also be fixedly coupled to the tether.

In one variation, pulling tether (1034), attaching tether (1034) to a most-proximal anchor, and cutting tether (1034) may be achieved using a termination device (not shown). The termination device may comprise, for example, a catheter advanceable over tether (1034) that includes a cutting member and a nitinol knot or other attachment member for attaching tether (1034) to the most-proximal anchor. The termination catheter may be advanced over tether (1034) to a location at or near the proximal end of tether-coupled anchors (1026). It may then be used to apply opposing force to the most-proximal anchor while tether (1034) is pulled to cinch the anchors and tighten the tissue. Attachment and cutting members may then be used to attach tether (1034) to the most-proximal anchor and cut tether (1034) just proximal to the most-proximal anchor. Such a termination device is only one possible way of accomplishing the pulling/cinching, attachment and cutting steps, and any other suitable device(s) or technique(s) may be used.

In some variations, it may be advantageous to deploy a first number of anchors (1026) along a first portion of the annular tissue, cinch the first anchors to tighten that portion of the annulus, move delivery device (1020) to another portion of the annulus, and deploy and cinch a second number of anchors (1026) along a second portion of the annulus. Such a method may be more convenient, in some cases, than extending delivery device (1020) around all or most of the circumference of the annulus, and may allow a shorter, more maneuverable housing (1022) to be used.

While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the appended claims.

1. A tether-anchor assembly comprising:
a tether comprising multiple filaments and having a distal end and an opening formed between the multiple filaments, wherein at least a portion of the multiple filaments between the distal end and the opening are heat-fused; and
an anchor with an eyelet segment located within the opening of the tether and a penetrating segment configured to penetrate a tissue.

2. The tether-anchor assembly of claim 1, wherein the tether further comprises a tether knot enclosing the eyelet segment and the distal end of the tether.

3. A tether-anchor assembly comprising:
a tether comprising a proximal end, a distal end, an elongate body therebetween, and an intrabody opening; and
an anchor with an eyelet segment located within the intrabody opening of the tether and a penetrating segment configured to penetrate a tissue.

4. The tether-anchor assembly of claim 3, wherein the tether is a multi-filament tether.

5. The tether-anchor assembly of claim 4, wherein the multi-filament tether comprises at least six strands.

6. The tether-anchor assembly of claim 5, wherein at least three strands are located on opposite sides of the intrabody opening.

7. The tether-anchor assembly of claim 3, wherein the tether is a monofilament tether.

8. The tether-anchor assembly of claim 3, wherein the tether is heat-treated at least between the distal end and the intrabody opening.

9. The tether-anchor assembly of claim 3, wherein the tether further comprises a stop region between the distal end and the intrabody opening.

10. The tether-anchor assembly of claim 9, wherein the stop region is a fused stop region.

11. The tether-anchor assembly of claim 4, wherein multiple filaments of the tether are at least partially fused between the distal end and the intrabody opening.

12. The tether-anchor assembly of claim 3, wherein the tether comprises polyethylene.

13. The tether-anchor assembly of claim 3, wherein the tether further comprises a tether knot.

14. The tether-anchor assembly of claim 13, wherein the tether knot encloses a portion of the anchor.

15. The tether-anchor assembly of claim 14, wherein the tether knot encloses a portion of the tether separate from the tether knot.

16. The tether-anchor assembly of claim 15, wherein the tether knot comprises a first loop and a second loop, and wherein the portion of the tether separate from the tether knot is enclosed in the second loop of the tether knot.

17. A method for making a tether-anchor assembly, comprising:
inserting a multi-filament tether into a protective tube;
heat-treating the multi-filament tether using the protective tube;
separating at least some filaments of the tether; and
passing an anchor through the separated filaments.

18. The method of claim 13, wherein separating at least some filaments of the tether comprises equally separating the filaments between a first bundle and a second bundle.

19. The method of claim 14, wherein a difference in the number of filaments in the first bundle and the second bundle are no greater than one filament.

20. The method of claim 17, further comprising forming a tether knot around the anchor.

21. The method of claim 17, further comprising forming a tether knot around a portion of the tether separate from the tether knot.

22. A tether-anchor assembly comprising:
a first anchor comprising a first eyelet region and a first penetrating region configured to penetrate a tissue;
a second anchor comprising a second eyelet region and a second penetrating region configured to penetrate the tissue; and
a tether slidably coupled to the first eyelet region and fixedly coupled to the second eyelet region via a knot assembly comprising a bowline knot.

23. The tether-anchor assembly of claim 22, wherein at least one of the first or second anchors is configured to self-expand.

24. The tether-anchor assembly of claim 22, wherein the knot assembly further comprises a figure-of-eight knot.

25. The tether-anchor assembly of claim 24, wherein the bowline knot is located between the second eyelet region and the figure-of-eight knot.

26. The tether-anchor assembly of claim 24, wherein the figure-of-eight knot is located between the bowline knot and the second eyelet region.

27. The tether-anchor assembly of claim 22, wherein the tether is made from a biodegradable material.

28. The tether-anchor assembly of claim 22, wherein the tether is made from a non-biodegradable material.

29. The tether-anchor assembly of claim 22, wherein the tether is a monofilament tether.

30. The tether-anchor assembly of claim 22, wherein the tether is a multifilament tether.

31. The tether-anchor assembly of claim 22, wherein at least a portion of the tether is coated with wax, silicone, silicone rubbers, PTFE, PBA, or ethyl cellulose.

32. The tether-anchor assembly of claim 22, wherein at least a portion of the tether comprises a surface that is textured to enhance the coefficient of friction of the surface.

33. The tether-anchor assembly of claim 22, wherein at least a portion of the tether is coated with one or more agents.

34. The tether-anchor assembly of claim 22, wherein at least a portion of the first anchor is coated with a Vascular Endothelial Growth Factor, Fibroblast Growth Factor, Platelet-Derived Growth Factor, Transforming Growth Factor Beta, insulin, insulin-like growth factors, estrogens, heparin, or Granulocyte Colony-Stimulating Factor.

35. The tether-anchor assembly of claim 22, wherein at least a portion of the second eyelet region comprises a surface that is textured to enhance the coefficient of friction of the surface.

36. A tether-anchor assembly comprising:
a first anchor comprising a first eyelet region and a first penetrating region configured to penetrate a tissue;
a second anchor comprising a second eyelet region and a second penetrating region configured to penetrate the tissue; and
a tether slidably coupled to the first eyelet region and fixedly coupled to the second eyelet region via a knot assembly;
wherein the knot assembly comprises a knot selected from a group consisting of a figure-of-eight knot, an eye splice and a back splice.
